(19) United States
(12) Patent Application Publication
(10) Pub. No.: US 2010/0016329 A1
(43) Pub. Date: Jan. 21, 2010

(54) XANTHINE-BASED CYCLIC GMP-ENHANCING RHO-KINASE INHIBITOR INHIBITS PHYSIOLOGICAL ACTIVITIES OF LUNG EPITHELIAL CELL LINE

(75) Inventor: Ing-Jun Chen, Kaohsiung City (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: KAOHSIUNG MEDICAL UNIVERSITY, Kaohsiung City (TW)

(21) Appl. No.: 12/176,607

(22) Filed: Jul. 21, 2008

(51) Int. Cl. A61K 31/496; C07D 473/04; A61P 35/00

(52) U.S. Cl. 514/252.16; 544/272

(57) ABSTRACT

The present invention provides a pharmaceutical composition including a compound of 7-[2-[4-(2-chlorophenyl)piperazinyl]-ethyl]-1,3-dimethylxanthine (KMUP-1), wherein the compound is a Rho-kinase inhibitor, and the pharmaceutical composition inhibits a physiological activity of a lung epithelial cell.

### FIELD OF THE INVENTION

The present invention relates to a compound of xanthine-based KMUP-1 performing inhibitory actions on physiological activities of cancer cells. More particularly, the present invention relates to a cGMP-enhancing Rho kinase (ROCK) inhibitor involving in down regulation of ROCK/VEGF by enhancing cGMP in lung epithelial cells, and thus inhibits cell migrations.

### BACKGROUND OF THE INVENTION

Lung epithelial cell proliferation, inflammation and migration are key events in the development of bronchia obstruction disease. Epithelial NOS/cGMP-signaling, involved in control of airway contractility and cell growth, deserves notice to resolve disease problems. Epithelial cells release various smooth muscle inhibitory mediators, for example, rapidly released nitric oxide (NO) from epithelium may influence adjacent smooth muscle cells contractility and growth in lung. Moreover, immunohistologic evidence has characterized the NO/cGMP-pathway in respiratory epithelium^{[1, 2]}.

KMUP-1 (7-[2-[4-(2-chloro benzene)piperazinyl]ethyl]-1,3-dimethylxanthine), a xanthine-based cyclic GMP (guanosine 3′,5′-cyclic monophosphate) enhancing ROCK inhibitor, has been found to relax tracheal contraction by activating soluble guanynyl cyclase (sGC) and epithelial nitric oxide synthase (NOS), leading to increase of cytosolic cGMP. Moreover, KMUP-1 inhibits TNFa-induced iNOS expression, involving sGC activation- and phosphodiesterase (PDE) inhibition-associated increase of cGMP/PKG in trachea smooth muscle^{[3]}. Accordingly, KMUP-1 is supposed to be involved in inhibiting proliferation, pro-inflammation and migration in H441 cells.

YC-1, a sGC activator with nitric oxide (NO)-independent cGMP-enhancing activity, displays anti-proliferation, anti-angiogenesis, anti-cancer and untoward pro-inflammatory effects in distinct cell types, and thus it is chosen as a positive control for comparison with KMUP-1 in the present invention. As to the anti-migration effect, ROCK is observed, when the epithelial cells are cultured with or without the ROCK inhibitor Y27632, since it involves in invasion and migration activity of cancer cells in the down stream of cGMP/PKG pathway.

With regard to anti-angiogenesis effect, vascular endothelial growth factor (VEGF) is known to be an important pro-angiogenic factor, necessary for tumor growth. Its expression is induced by a number of stimuli, including hypoxia, evidenced by expression of hypoxic induced factor 1 (HIF-1), a hypoxia-activated transcription factor that can regulate VEGF gene. In comparison, since YC-1 is a representative one of NO-independent cGMP enhancer, it also has predominately functions in vascular system and displays inhibitory effect on VEGF and HIF-1a expression in Hep3B cells^{[6, 7]}.

On the other hand, expression of the cyclin-dependent kinase (CDK)-inhibitory proteins p21 and p27 which associated with anti-proliferation activities is observed whether the above two proteins are increased in cGMP-pathway. Besides p21 and p27, phosphorylation of another protein kinase p38 is inspected in this invention. In contrast, p38 plays an important role in inflammatory cells, proliferation of airway structural cell and cell survival^{[8, 9]}. Additionally, the apoptotic signaling Bax/Bcl-2/caspase 3, accompanied by p21 and p27 expression in cell cycle, is also analyzed in the present invention for understanding the apoptosis of cancer cells.

In this invention, we characterized the effects of KMUP-1 on proliferation, migration and pro-inflammation in H441 cells, including eNOS/sGC/PKG and apoptotic signaling Bax/Bcl-2/caspase 3, accompanied by p21 and p27 expression in cell cycle, particularly ROCK II/VEGF/HIF-1a expression in hypoxia.

### SUMMARY OF THE INVENTION

This invention relates to the inhibitory effects on lung epithelial cells of KMUP-1, which upon laboratory testing have been proven that it reveals promising effects on anti-proliferation, anti-proinflammation and anti-metastasis of cancer cells through affecting the expressions of cGMP-related eNOS/sGC/PDE5A, apoptotic signal Bax/Bcl-2/caspase 3 and ROCKII/VEGF/HIF-1a in hypoxic state.

The present invention provides a pharmaceutical composition, which comprises a compound of 7-[2-[4-(2-chlorophenyl)piperazinyl]-ethyl]-1,3-dimethylxanthine(KMUP-1), wherein the compound is a Rho-kinase inhibitor, and the pharmaceutical composition inhibits a physiological activity of a lung epithelial.

Preferably, the pharmaceutical composition further comprises a pharmaceutically acceptable carrier.

Preferably, the physiological activity is one selected from a group consisting of a proliferation activity, a migration activity, a pro-inflammatory activity and a combination thereof.

Preferably, the migration activity is a metastasis activity of a cancer cell.

Preferably, the physiological activity results from cGMP increasing activity and ROCK inhibition in the lung epithelial cell.

In another aspect, the present invention provides a method for inhibiting a physiological activity of a lung epithelial cell, which comprises a step of administrating a pharmaceutically effective amount of a compound of 7-[2-[4-(2-chlorophenyl)piperazinyl]-ethyl]-1,3-dimethylxanthine(KMUP-1) to a mammal in need, wherein the compound is a Rho-kinase inhibitor and being synthesized from xanthine.

Preferably, the method further comprises a pharmaceutically effective carrier.

Preferably, the physiological activity is one selected from a group consisting of a proliferation activity, a migration activity, a pro-inflammatory activity and a combination thereof.

Preferably, the migration activity is a metastasis activity of a cancer cell.

Preferably, the physiological activity is inhibited by cGMP-enhancing and ROCK inhibitory property of the compound.

Furthermore, the present invention provides a method for preparing a pharmaceutical composition, wherein the pharmaceutical composition contains a compound of 7-[2-[4-(2-chlorophenyl)piperazinyl]-ethyl]-1,3-dimethylxanthine(KMUP-1).

Preferably, the pharmaceutical composition has an inhibitory effect on one physiological activity of a lung epithelial cell selected from a group consisting of a proliferation, a migration, a pro-inflammatory and a combination thereof.

Preferably, the migration activity is a metastasis activity of a cancer cell.

Preferably, the pharmaceutical composition further contains a pharmaceutically acceptable carrier.

The above aspects and advantages of the present invention will become more readily apparent to those ordinarily skilled in the art after reviewing the following detailed description and accompanying drawings, in which:

### BRIEF DESCRIPTION OF THE DRAWINGS

FIGS. 1(A) and 1(B) show the inhibitory effect of different concentrations of KMUP-1 (1.0, 10 and 100 μM) on the survival rate of H441 cell in the normoxia (FIG. 1A) and hypoxia (FIG. 1B) state, wherein the circle represents the results of the control group, the triangle represents the results of 1.0 μM KMUP-1, the empty triangle represents the results of 10 μM KMUP-1 and the square represents the results of 100 μM KMUP-1, respectively;

FIG. 2 shows the effect of different concentrations of KMUP-1 (0.01, 0.1, 1.0, 10 and 100 μM) on cell cycle distribution proportions (%), wherein FCS and vehicle represent the culturing medium with fetal calf serum and vehicle control, respectively, and the symbols Go/G1, S and G2/M represent the resting/Gap1, synthesis and Gap2/mitosis phases during a cell cycle;

FIGS. 3(A)-3(C) show the effect of KMUP-1 (1.0 μM) on the percentages of each phase in a cell cycle from 6 hrs to 72 hrs, wherein SF and 10% FCS represent the serum starvation culturing medium and medium supplemented with 10% FCS, respectively;

FIG. 4 is cell cycle area graphs analyzed by a flow cytometry showing the effect of 100 μM KMUP-1 on cell cycle distribution as compared with the control and the vehicle control therof;

FIGS. 5(A) and 5(B) show the eNOS expression of H441 cells treated with (FIG. 5B) or without (FIG. 5A) KMUP-1(10 μM) in normoxia and hypoxia at 12, 24, 48 and 72 hrs, wherein the expression of eNOS is normalized with that of β-actin, the slash bar represents the eNOS expression at 0 hr while the cross bars and white bars respectively represent the eNOS expressions under normoxia and hypoxia at different times, and the symbols * and ** represent the significant of p<0.05 and p<0.01 compared with 0 hr., respectively, and the symbols # and ## represent the significant of p<0.05 and p<0.01 comparison between normoxia and hypoxia at indicated time, respectively;

FIG. 6 shows the effects of different concentrations of KMUP-1 (0.001, 0.01, 0.1, 1.0, and 10 μM) on the expression of eNOS in normoxia within 48 hrs, wherein the expression of eNOS is normalized with that of β-actin, the slash bar represents the control group while the empty bars represent the KMUP-1 groups;

FIGS. 7(A) and 7(B) show the effects of KMUP-1 on the expression of eNOS in normoxia and hypoxia when the cells are pretreated with a NOS inhibitor, L-NAME (100 μM) 30 mins before KMUP-1, wherein the slash bars represent the groups without pretreatment and the empty bars represent the groups with a L-NAME pretreatment, the concentrations of KMUP-1 in FIGS. 7(A) and 7(B) are 0.1 μM and 1.0 μM, respectively;

FIG. 8 shows the effects of KMUP-1 (10, 100 μM) on sGCa expression in H441 cells under normoxic and hypoxic conditions, wherein the symbol SF represents the cell cultured in serum free medium, and the slash bars and empty bars represent the normoxia and hypoxia states, respectively;

FIG. 9 shows the effects of KMUP-1 on PKG expression in H441 cells under normoxic and hypoxic conditions, wherein the symbol SF represents the cell cultured in serum free medium, and the slash bars and empty bars represent the normoxia and hypoxia states, respectively;

FIG. 10 shows the effects of KMUP-1 (1 μM) on HIF-1a protein expression in H441 cells following exposure to hypoxia from 3 hrs to 72 hrs, wherein the slash bars and empty bars represent the control groups and KUMP-1 groups, respectively;

FIG. 11 shows the effects of KMUP-1 (1 μM) on VEGF expression in H441 cells following exposure to hypoxia from 3 hrs to 72 hrs, wherein the slash bars and empty bars represent the control groups and KUMP-1 groups, respectively;

FIG. 12 shows the results of the inhibitory effects of KMUP-1 on HIF-1a and VEGF expression after L-NAME (100 μM) pretreatment for 30 mins, wherein the cells are treated with 1 μM KMUP-1 for 24 hrs;

FIGS. 13(A) and 13(B) show the effects of different concentrations of KMUP-1 (0.01, 0.1, 1.0, 10 and 100 μM) on the expression of HIF-1a (FIG. 13A) and VEGF (FIG. 13B) for 24 hrs in hypoxia, wherein the slash bar represents the control group while the empty bars represent the KMUP-1 groups;

FIGS. 14(A) and 14(B) show the expression of HIF-1a (FIG. 14A) and VEGF (FIG. 14B) under different treatments for 24 hrs in hypoxia, including vehicle control, 1.0 μM KMUP-1, 1.0 μM YC-1, 100 μM SNP and 100 μM IBMX;

FIG. 15 shows the effects of KMUP-1 (10, 100 μM) on ROCKII protein expression in H441 cells under normoxic and hypoxic conditions, wherein the symbol SF represents the cell cultured in serum free medium, and the slash bars and empty bars represent the normoxia and hypoxia states, respectively;

FIG. 16 shows the effects of KMUP-1 (10 μM) on the expression of Rho kinase protein in normoxia and hypoxia states after pretreatment with Rp-8-CPT-cGMP (10 μM) for 30 mins, wherein the cells are treated with KMUP-1 for 48 hrs, and the slash bars and empty bars represent the normoxia and hypoxia states, respectively;

FIGS. 17(A) and 17(B) show the relative distances across the wound width of H441 cells after culturing with 10% fetal bovine serum, serum free medium, KMUP-1 (1-100 μM) and the ROCK inhibitor Y27632 (10 μM) in normoxia (FIG. 17A) and hypoxia (FIG. 17B) states for 48 hrs;

FIGS. 18(A) and 18(B) show the effects of KMUP-1 on p21 expression in H441 cells under normoxic and hypoxic conditions after pretreated with (FIG. 18B) or without (FIG. 18A) a cGMP antagonist, Rp-8-CPT-cGMP (10 μM) for 30 mins, wherein the symbol CTL represents the control group, the symbol SF represents the cell cultured in serum free medium, and the lash bars and empty bars represent the normoxia and hypoxia states, respectively;

FIGS. 19(A) and 19(B) show the results of KMUP-1 (100 μM) on the expression of p27 in normoxia and hypoxia states after pretreatment with (FIG. 19B) or without (FIG. 19A) a cGMP antagonist, Rp-8-CPT-cGMP (10 μM) for 30 mins, wherein the symbol CTL represents the control group, the symbol SF represents the cell cultured in serum free medium, and the slash bars and empty bars represent the normoxia and hypoxia states, respectively;

FIGS. 20(A) and 20(B) show the effects of KMUP-1 (10, 100 μM) on Bax (FIG. 20A) and Bcl-2 (FIG. 20B) expressions in H441 cells under normoxic and hypoxic conditions, wherein the symbol SF represents the cell cultured in serum free medium, and the slash bars and empty bars represent the normoxia and hypoxia states, respectively;

FIG. 21 shows the effects of KMUP-1 (10, 100 μM) on Bax/Bcl-2 ratio in H441 cells under normoxic and hypoxic conditions, wherein the symbol SF represents the cell cultured in serum free medium, and the slash bars and empty bars represent the normoxia and hypoxia states, respectively;

FIG. 22 shows the effects of KMUP-1 (100 μM) on the Bax/Bcl-2 ratio in normoxia and hypoxia states after pretreating with a NOS inhibitor, L-NAME (100 μM) for 30 mins, wherein the cells are treated with KMUP-1 for 48 hrs, and the slash bars and empty bars represent the normoxia and hypoxia states, respectively;

FIG. 23 shows the effects of KMUP-1 (100 μM) on the Bax/Bcl-2 ratio in normoxia and hypoxia states after pretreating with a cGMP antagonist, Rp-8-CPT-cGMP (10 μM) for 30 mins, wherein the cells are treated with KMUP-1 for 48 hrs, and the slash bars and empty bars represent the normoxia and hypoxia states, respectively;

FIG. 24 shows the effects of KMUP-1 (1, 10, 100 μM) on procaspase-3/caspase-3 ratio in H441 cells under normoxic and hypoxic conditions, wherein the slash bars and empty bars represent the normoxia and hypoxia states, respectively;

FIG. 25 shows the effects of KMUP-1 on PDE5A expression in H441 cells under normoxic and hypoxic conditions, wherein the symbol SF represents the cell cultured in serum free medium, and the slash bars and empty bars represent the normoxia and hypoxia states, respectively;

FIG. 26 shows the effects of different concentrations of KMUP-1 (1.0, 10 and 50 μM) on the expression of PDE5A in H441 cells treated with a PDE5A enhansor, U46619 (5 μM), in normoxia and hypoxia states, wherein the slash bars and empty bars represent the normoxia and hypoxia states, respectively;

FIG. 27 shows the effects of KMUP-1 (10, 100 μM) on phosphate p-38/total p38 ratio in H441 cells under normoxic and hypoxic conditions, wherein the symbol SF represents the cell cultured in serum free medium, and the slash bars and empty bars represent the normoxia and hypoxia states, respectively;

FIG. 28 shows the effects of different concentrations of KMUP-1 (1-100 μM) on the expression of TNF-a-induced iNOS in normoxia and hypoxia, wherein the slash bars and empty bars represent the normoxia and hypoxia states, respectively.

### DETAILED DESCRIPTION OF THE PREFERRED EMBODIMENT

The invention is described more specifically with reference to the following embodiments. It is to be noted that the following descriptions of preferred embodiments of this invention are presented herein for the purpose of illustration and description only; it is not intended to be exhaustive or to be limited to the precise form disclosed.

The present provides a pharmaceutical composition comprising a compound of KMUP-1 (7-[2-[4-(2-chlorobenzene)piperazinyl]ethyl]-1,3-dimethylxanthine). The structure and synthetic method of KMUP-1 of the present invention have been disclosed in U.S. Pat. No. 6,979,687, and are not described hereafter.

The Pharmacological Activities of the Compounds of the Present Invention have been Proven by the Following Pharmacological Experiments.

Cell Survival Rate

Human lung adenocarcinoma NCI-H441 cell lines are obtained from the American Type Culture Collection and are cultured in RPMI 1640 medium supplemented with 2 mM glutamine, penicillin/streptomycin and 10% fetal calf serum. Cells are grown in a humidified atmosphere containing 5% CO₂ at 37° C. under normoxia (20% O₂) and hypoxia (1% O₂). To achieve hypoxia, a pre-analyzed gas mixture (95% N₂-5% CO₂) was infused into an air chamber.

For the test of cell survival or proliferation, H441 cells are cultured in 24-well plates (10⁵ cells/well) and incubated with different concentrations of KMUP-1 for various lengths of time followed by MTT assay. All data are expressed as the mean±S.E., n=4. Statistical differences were determined by independent and paired Student's t-test in unpaired and paired samples, respectively. Whenever a control group was compared with more than one treated group, the one-way or two-way ANOVA was used. When the ANOVA manifested a statistical difference, results were further analyzed with Dunnett's or Tukey test. A probability value (p-value) less than 0.05 was considered to be significant. Analysis of data and plotting of figures were done with on SigmaStat: Version 2.03 and SigmaPlot: Version 8.0 (Systat Software, Point Richmond, Calif.) and run on an IBM-compatible computer.

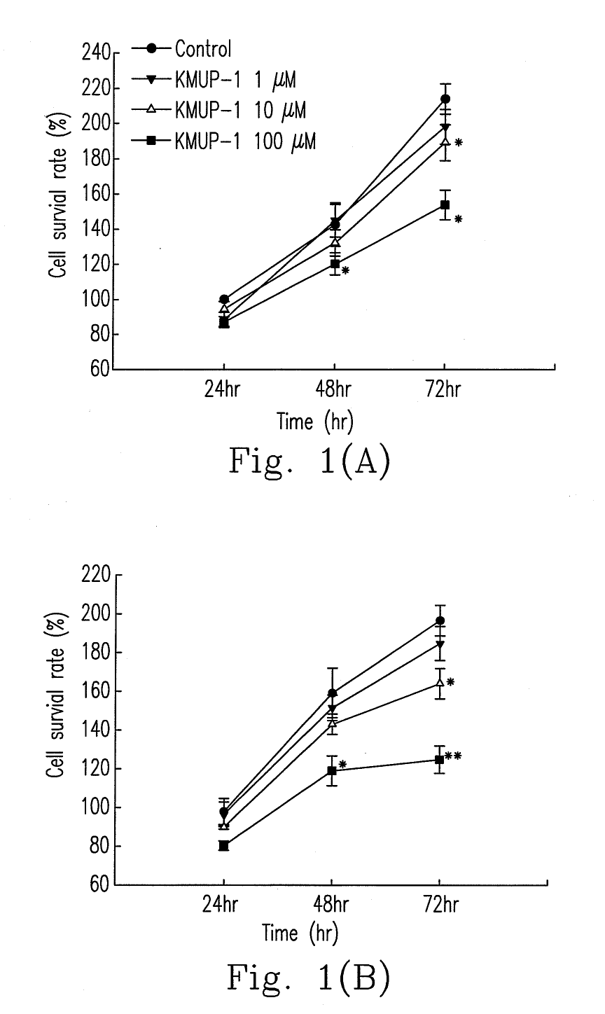

Please refer to FIGS. 1(A) and 1(B) show the inhibitory effect of different concentrations of KMUP-1 (1.0, 10 and 100 μM) on the survival rate of H441 cell in the normoxia (FIG. 1A) and hypoxia (FIG. 1B) state for 24 hrs, 48 hrs and 72 hrs. In normoxia and hypoxia condition, KMUP-1 (10, 10, 100 μM) inhibits the survival rate of cultured H441 cell line. As FIGS. 1(A) and 1(B) show, KMUP-1 significantly inhibits the survival rate of H441 cells at higher concentrations (≧10 μM).

Cell Cycle Distribution

Cells are harvested by trypsinization, washed with PBS, and re-suspended in 75% ethanol in PBS and kept at 4° C. for at least 30 min. Before analysis, cells are washed again with PBS, resuspended, and incubated for 30 min in propidium iodide staining solution containing 0.05 mg/ml propidium iodide, 1.0 mM EDTA, 0.1% TritonX-100 and 1 mg/ml RNase A in PBS. The suspension is then passed through a nylon mesh filter and analyzed on a flow cytometry (Coulter Epics XL-MCL, Beckman Coulter, USA).

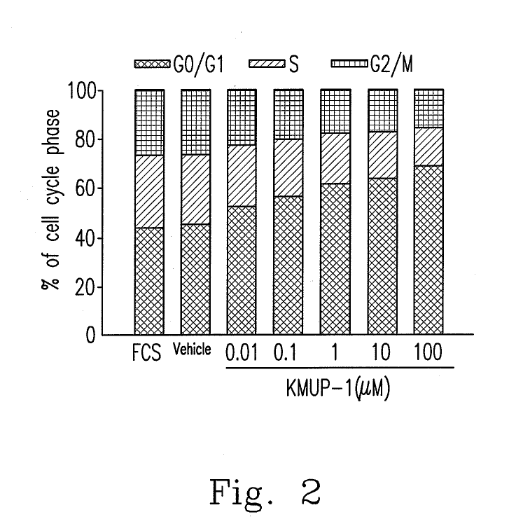

Please refer to FIG. 2, which shows the effects of different concentrations of KMUP-1 (0.01, 0.1, 1.0, 10 and 100 μM) on cell cycle distribution proportions (%). In normoxia condition, flow cytometric analysis demonstrats effects of KMUP-1 on progression of cell cycle. Cell cycle distribution is affected by KMUP-1 (0.01, 0.1, 1.0, 10, 100 μM) concentration-dependently. The result of FIG. 2 shows that the percentage of Go/G1 phase is increased with the increasing KMUP-1 concentrations, and that of S phase and G2/M phase are decreased with the increasing KMUP-1 concentrations.

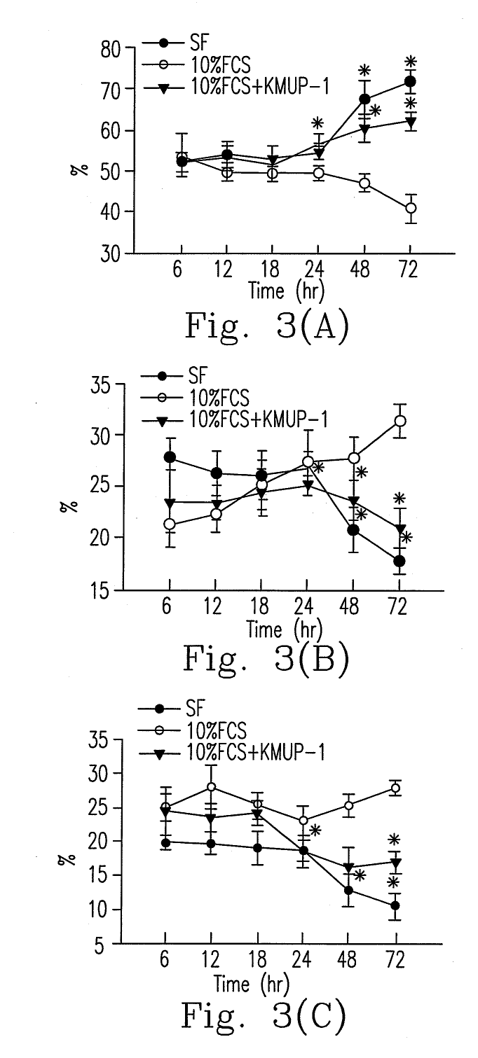

Please refer to FIGS. 3(A) to 3(C), which show the effect of KMUP-1 (1.0 μM) on the percentages of each phase in a cell cycle from 6 hrs to 72 hrs. As FIGS. 2, 3(A), 3(B) and 3(C) illustrate, KMUP-1 (100 μM) significantly arrests cell cycle at G0/G1 phase for 72 hrs.

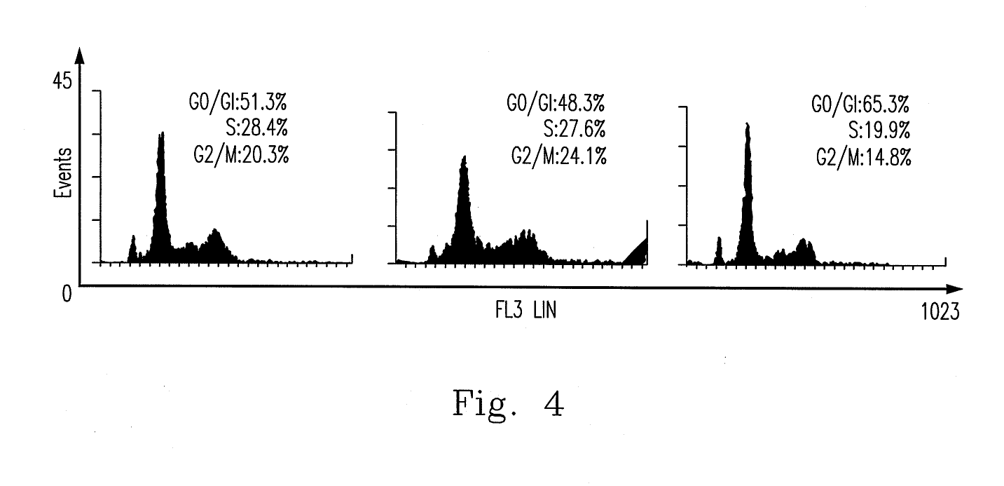

Please refer to FIG. 4, which is cell cycle area graphs analyzed by a flow cytometry showing the effects of 100 μM KMUP-1 on cell cycle distribution as compared with the control and the vehicle control. As shown in FIG. 4, KMUP-1 (100 μM) significantly arrests cell cycle at G0/G1 phase for 72 hr.

NOS, sGC and PKG Expression

To determine the expression levels of eNOS, iNOS, sGC, PKG, HIF-1a, VEGF, ROCKII, p38, Bax, Bcl-2 and cyclin-dependent kinase (CDK)-inhibitory proteins p21 and p27 in H441 cell line in this invention, the total proteins are extracted and Western blot analyses are performed as described below. Briefly, H441 cells are cultured in 10-cm dishes. After reaching subconfluence, the cells are rendered quiescent and then treated with various time or concentrations of KMUP-1. In some experiments, cells were pretreated with specific inhibitors as indicated, then followed by KMUP-1. Measurement of iNOS was performed in the presence of TNF-a (100 ng/ml) for 30 min after pre-incubation of cells with KMUP-1. After incubation, the cells are washed with PBS (pH 7.4), incubated with extraction buffer (Tris 10 mM, pH 7.0, NaCl 140 mM, PMSF 2 mM, DTT 5 mM, NP-40 0.5%, pepstatin A 0.05 mM and leupeptin 0.2 mM) with gentle shaking, and then centrifuged at 12,500 g for 30 min. The cell extract is then boiled in a ratio of 1:1 with sample buffer (Tris 100 mM, pH 6.8, glycerol 20%, SDS 4% and bromophenol blue 0.2%). Electrophoresis is performed using 10% SDS-polyacrylamide gel (2 hr, 100 V, 40 mA, 50 mg protein per lane). Separated proteins are transferred to PVDF membranes (90 min, 100 V), treated with 5% fat-free milk powder to block the nonspecific IgGs, and incubated for 1 hr with specific antibody. The blot was then incubated with anti-mouse or -goat IgG linked to alkaline phosphatase (1:1000) for 1 hr. Protein bands were visualized by enhanced chemiluminescence reagents (GE Healthcare Bio-Sciences Corp., Piscataway, N.J.).

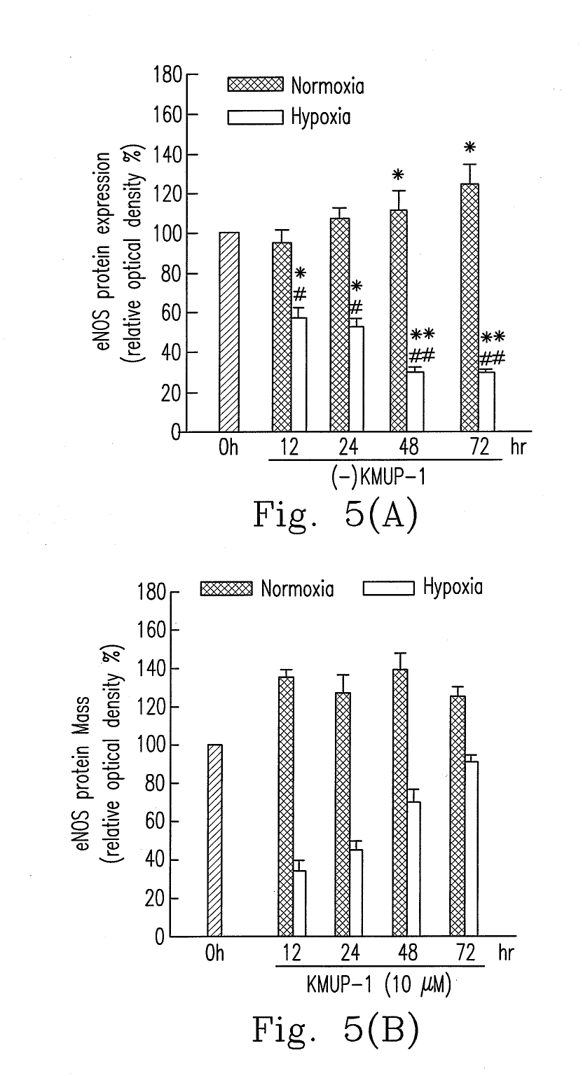

Please refer to FIGS. 5(A) and 5(B), which show the eNOS expression of H441 cells treated with (FIG. 5B) or without (FIG. 5A) KMUP-1(10 μM) in normoxia and hypoxia at 12, 24, 48 and 72 hrs. In normoxia, KMUP-1 (10 μM) stimulates the expression of eNOS in a time-dependent manner in H441 cell line. The maximal eNOS expression is achieved at 48 hrs. In hypoxia, the protein expression of eNOS in H441 cells is time-dependently decreased, but sharply up-regulated by KMUP-1.

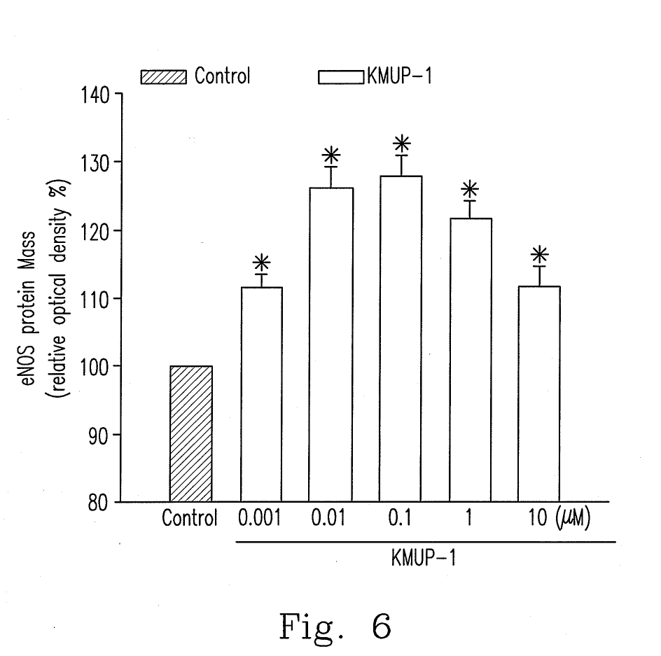

Please refer to FIG. 6, which shows the effects of different concentrations of KMUP-1 (0.001, 0.01, 0.1, 1.0, and 10 μM) on the expression of eNOS in normoxia within 48 hrs. As shown in FIG. 6, the induction of eNOS expression by KMUP-1 is also dose-dependent within 48 hr.

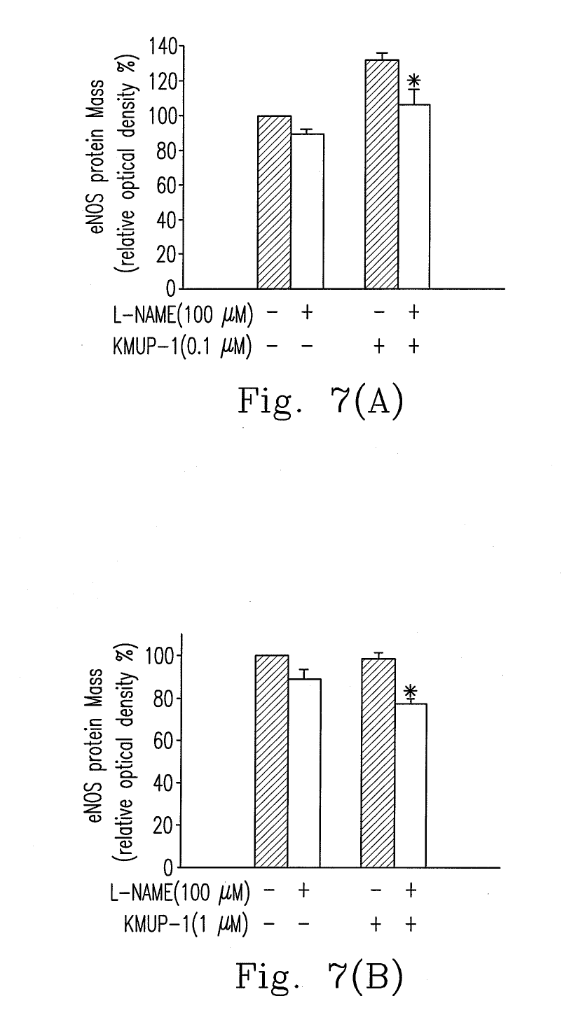

Please refer to FIGS. 7(A) and 7(B), which show the effects of KMUP-1 on the expression of eNOS in normoxia and hypoxia states when the cells are pretreated with a NOS inhibitor, L-NAME (100 μM) 30 mins before KMUP-1. As FIG. 7(A) shows, increases of eNOS protein expression in both conditions by KMUP-1 are reduced, but not completely, by pretreatment with a NOS inhibitor L-NAME (100 μM).

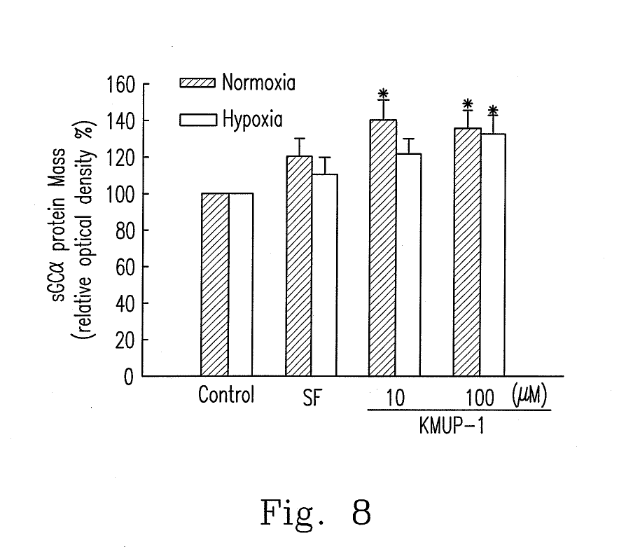

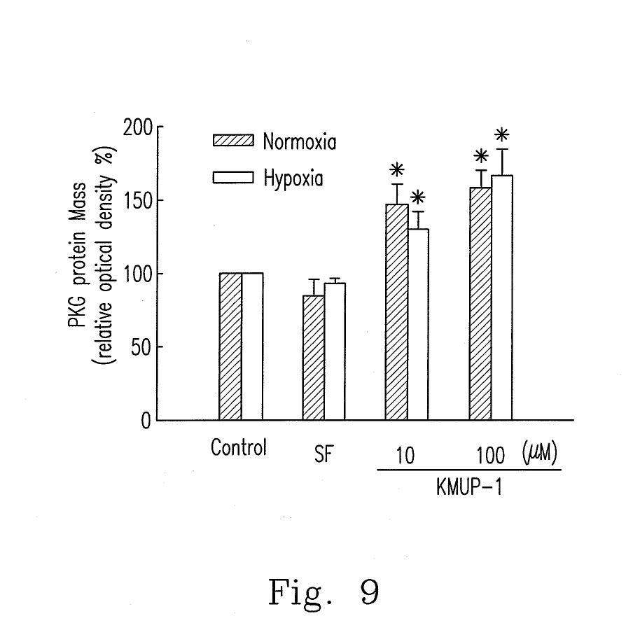

Please refer to FIGS. 8-9, which show the effects of KMUP-1 (10, 100 μM) on sGC/PKG signaling pathway in H441 cells under normoxic and hypoxic conditions. Cells are incubated with or without KMUP-1 for 48 hrs under both normoxic and hypoxic conditions. As FIG. 8 shows, sGCa expression in H441 cells is increased by KMUP-1 (10, 100 μM) in both conditions. Expression of PKG in H441 cells were significantly increased by KMUP-1 (10, 100 μM) from control to 146.8±13.9%, 157.9±12.1% in normoxia and 130.1±11.7%, 166.6±18.0% in hypoxia (FIG. 9).

A previous report demonstrated that activation of PKG is sufficient to induce growth inhibition and apoptosis and also to inhibit cell migration in human cancer cells^{[10]}. KMUP-1 (10, 100 μM) enhanced sGC and PKG expression in H441 cells both in normoxia and hypoxia, suggesting the ability to stimulate apoptosis and to inhibit cell growth and migration. Generally, activation of eNOS and sGC contributes to cGMP-mediated upregulation of PKG expression. Based on the above results, we further suggest that suppression of cell growth by KMUP-1 at higher concentration is related to long term increase of NO/peroxynitrate and cGMP/PKG.

HIF-1a, VEGF and ROCKII Signaling

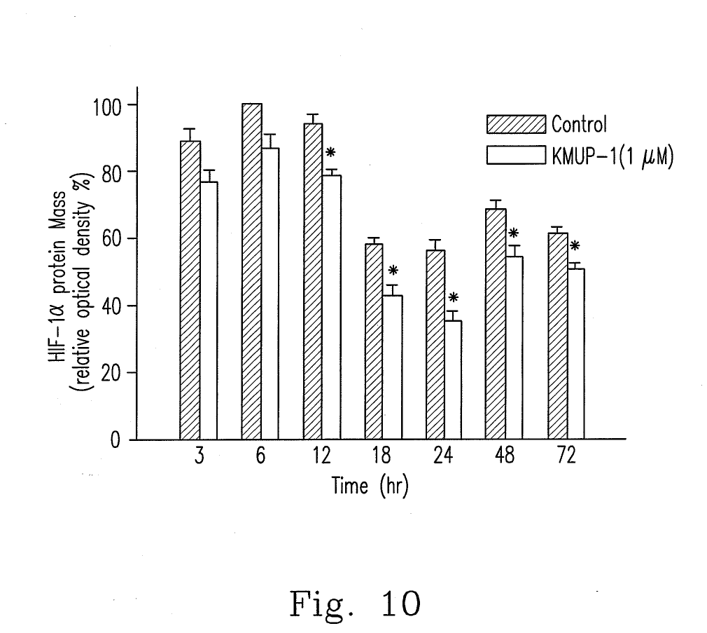

Please refer to FIG. 10, which shows the effects of KMUP-1 (1 μM) on HIF-1a protein expression in H441 cells following exposure to hypoxia from 3 hrs to 72 hrs. Significant expression of HIF-1a appears following exposure to hypoxia for 3 hrs and achieves the maximum during at 6 and 12 hrs. After exposure to hypoxia for 18˜24 hrs, the expression of HIF-1a protein is fast decreased. KMUP-1 (1 μM) inhibits HIF-1a expression at 12 hrs and achieved the maximum at 24 hrs.

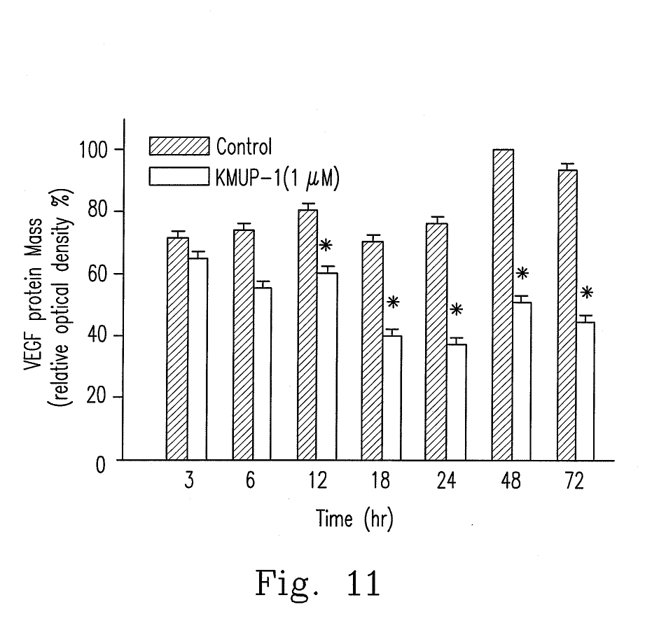

Please refer to FIG. 11, which shows the effects of KMUP-1 (1 μM) on VEGF expression in H441 cells following exposure to hypoxia from 3 hrs to 72 hrs. VEGF expressions are also increased in hypoxic condition, and the inhibitory effects of KMUP-1 (1.0 μM) at 12, 18, 24, 48 and 72 hrs are 59.9±3.0%, 39.6±4.4%, 37.0±5.1%, 50.7±3.5% and 44.2±3.3%, respectively.

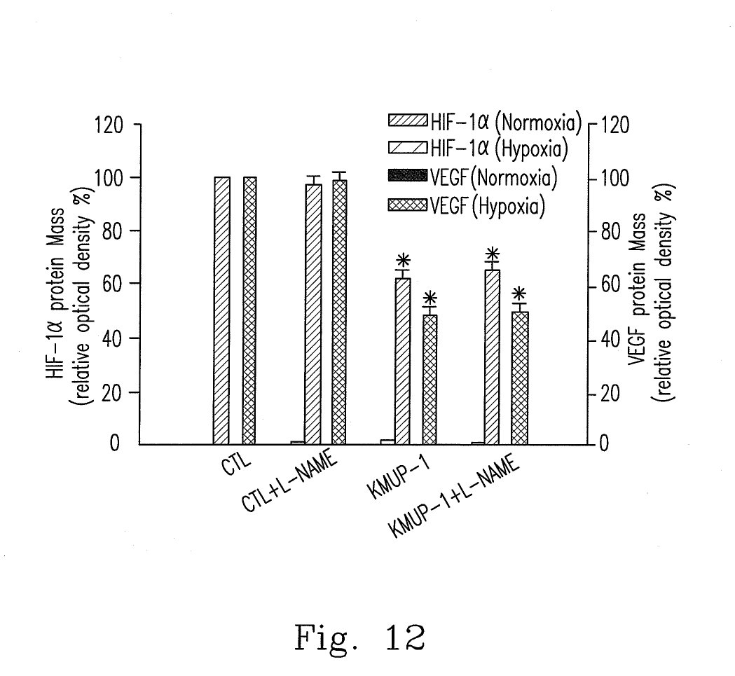

Please refer to FIG. 12, which shows the results of the inhibitory effects of KMUP-1 on HIF-1a and VEGF expression after L-NAME (100 μM) pretreatment for 30 mins. It is observed that the HIF-1a is not significantly expressed in normoxia, but significantly observed in hypoxic condition. KMUP-1 affects the expression of HIF-1a, . . . but the action is not observed by pretreatment with L-NAME. Combination of KMUP-1 with L-NAME do not show any further inhibition on HIF-1a protein in hypoxic state.

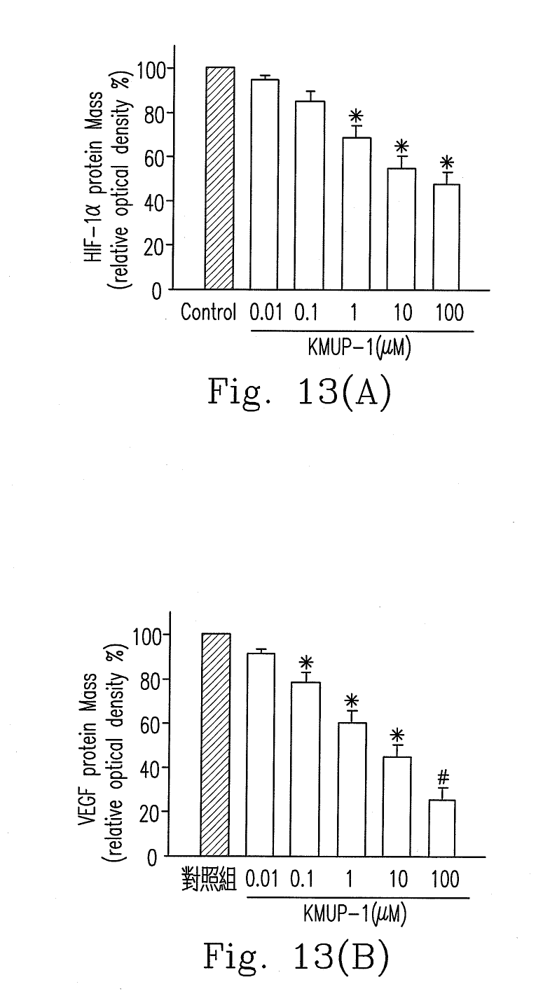

Please refer to FIGS. 13(A) and 13(B), which show the effects of different concentrations of KMUP-1 (0.01, 0.1, 1.0, 10 and 100 μM) on the expression of HIF-1a (FIG. 13A) and VEGF (FIG. 13B) for 24 hrs in hypoxia. As FIGS. 13(A) and 13(B) show, the expressions of HIF-1a and VEGF are concentration-dependently inhibited by KMUP-1 (0.01˜100 μM).

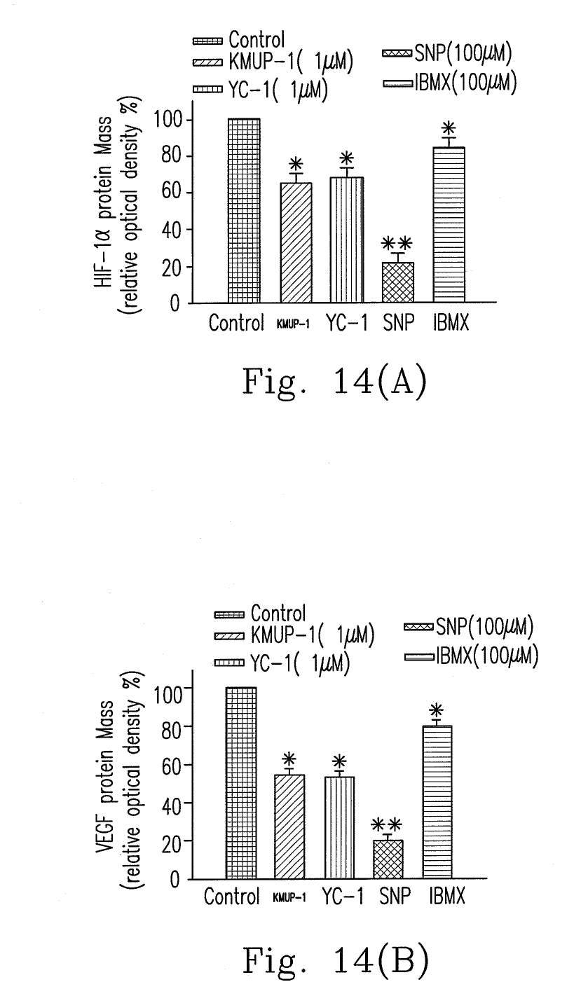

Please further refer to FIGS. 14(A) and 14(B), which show the expression of HIF-1a (FIG. 14A) and VEGF (FIG. 14B) under different treatments for 24 hrs in hypoxia, including control, 1.0 μM KMUP-1, 1.0 μM YC-1, 100 μM SNP and 100 μM IBMX. According to the data in FIG. 14(A), HIF-1a protein is inhibited by various treatments as follows (% of control): 34.8±2.3% (KMUP-1, 1.0 μM), 31.6±3.4% (YC-1, 1.0 μM), 78.7±3.1% (SNP, 100 μM), 15.9±5.9% (IBMX, 100 μM), respectively. Similarly, the data in FIG. 14(B) show VEGF protein is inhibited by various treatments as follows (% of control): 45.5±3.1% (KMUP-1, 1.0 μM), 47.2±4.4% (YC-1, 1.0 μM), 80.1±2.8% (SNP, 100 μM), 20.2±4.7% (IBMX, 100 μM).

Based on the above results, KMUP-1 (1-100 μM) concentration-dependently inhibits hypoxia-induced expression of VEGF and HIF-1a (FIGS. 13A and 13B). In the other hand, KMUP-1 does not show any significant VEGF expression at concentrations <1.0 μM in normoxia, indicating that it has no pro-angiogenic ability (FIG. 12). Taken together, KMUP-1 not only surmounts the possible NO-mediated VEGF production in hypoxia, but also has a potent anti-proliferation and/or anti-angiogenesis effect. In the present invention, KMUP-1 inhibits the expression of VEGF and HIF-1a in hypoxia, and reveals anti-angiogenesis and anti-tumor activities targeting those hypoxic protein markers.

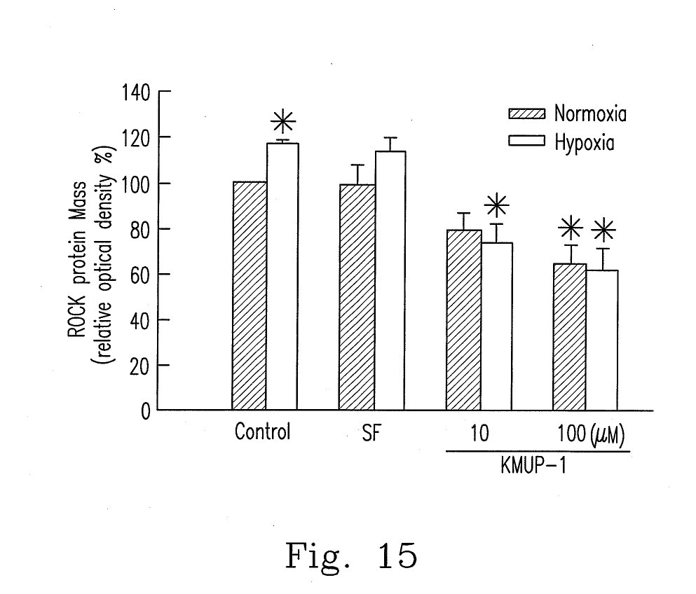

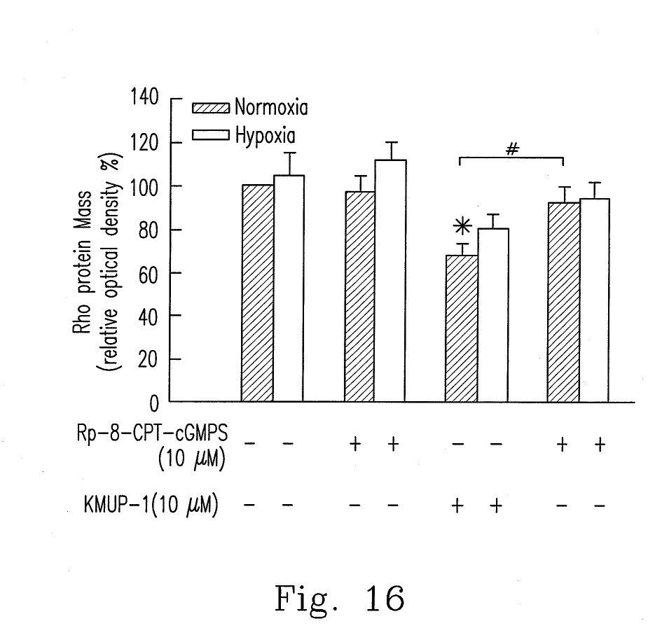

Please refer to FIG. 15, which shows the effects of KMUP-1 (10, 100 μM) on ROCKII protein expression in H441 cells under normoxic and hypoxic conditions. It is found that the expression of ROCKII is concentration-dependently decreased by KMUP-1. However, the above result is reversed by cGMP antagonist, Rp-8-CPT-cGMP (10 μM), as FIG. 16 shows.

Cell Migration and ROCKII Inhibition

H441 cells are cultured in 6-well plates until cells are 90% confluent. Wounds are produced by scraping the cell monolayer with a pipette tip across the diameter of the well and washed four times with medium to remove cell debris. Then, cells are treated with KMUP-1 and Y-27632 at 10 μM for 24 and 48 hrs. The wound edge is viewed and photographed under a microscope (Eclipse TS 100, Nikon). Wound width measurements are collected from two different (maximal and minimal) locations in the same well and averaged as one measurement.

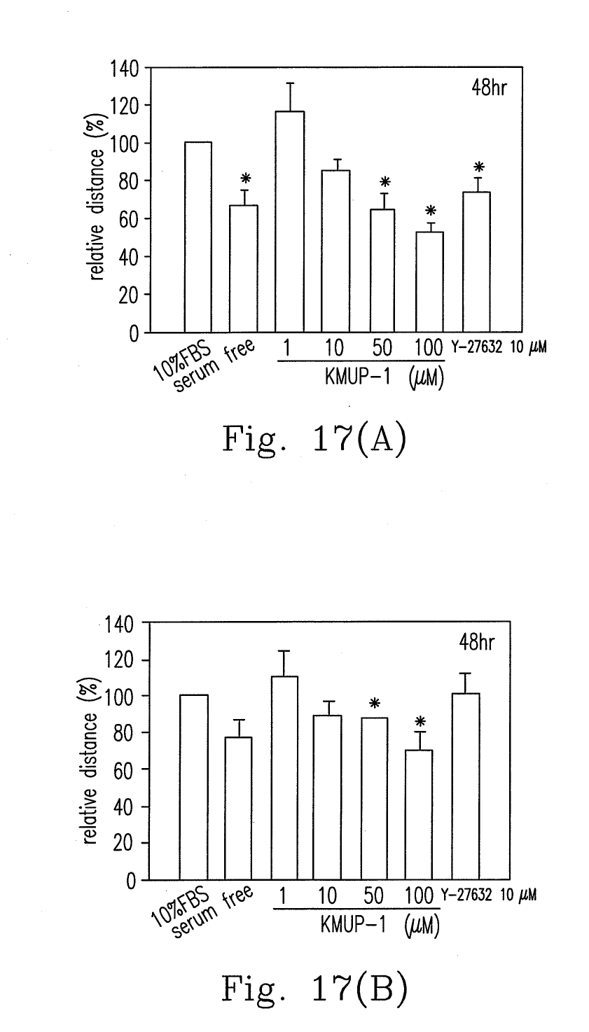

In correspond to the results of ROCKII expression, a cell migration experiment is used in this invention so as to prove the effect of KMUP-1 on migration activity of cancer cells, i.e. metastasis activity. Please refer to FIGS. 17(A) and 17(B), which show the relative distances across the wound width of H441 cells after culturing with 10% fetal bovine serum, serum free medium, KMUP-1 (1-100 μM) and the ROCK inhibitor Y27632 (10 μM) in normoxia (FIG. 17A) and hypoxia (FIG. 17B) states for 48 hrs. As FIGS. 17(A) and 17(B) show, KMUP-1 inhibits the migration activity of H441 lung epithelial cells in normoxic and in hypoxic condition. Moreover, KMUP-1 (≧50 μM) significantly reduces the migration of H441 cells across the wound width of culture after culturing for 48 hrs, and the effect is more prominent in normoxia than in hypoxia. Comparing the inhibitory effect of KMUP-1 on ROCKII with the ROCKII inhibitor Y27632, Y27632 (10 μM) also shows the inhibition activity in normoxia, but not in hypoxia. It is known that migration of epithelial cancer cell is suggested to increase the risk of cancer metastasis. Accordingly, in the resent invention, inhibition on ROCKII expression by KMUP-1 provides the anti-metastasis potential in lung epithelial cells.

p21 and p27 Expression

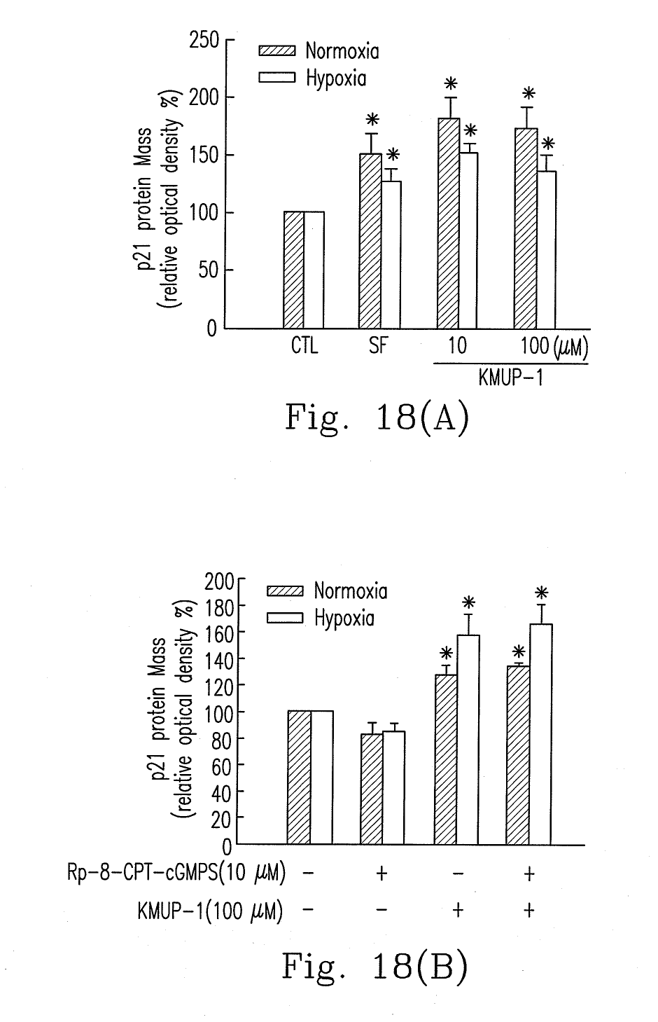

Please refer to FIGS. 18(A) and 18(B), which the effects of KMUP-1 on p21 expression in H441 cells under normoxic and hypoxic conditions after pretreated with (FIG. 18B) or without (FIG. 18A) a cGMP antagonist, Rp-8-CPT-cGMP (10 μM) for 30 mins. Cells are incubated with or without KMUP-1 for 48 hrs under normoxic and hypoxic condition. p21 expression is increased by KMUP-1 (10, 100 μM) compared to control (considered as 100%) as following: 181.2±17.6%, 172.9±18.1% (normoxia) and 151.7±7.7%, 135.1±14.7% in hypoxia (FIG. 18A). However Rp-8-CPT-cGMPS (10 μM) could not inhibit KMUP-1-induced p21 protein (FIG. 18B).

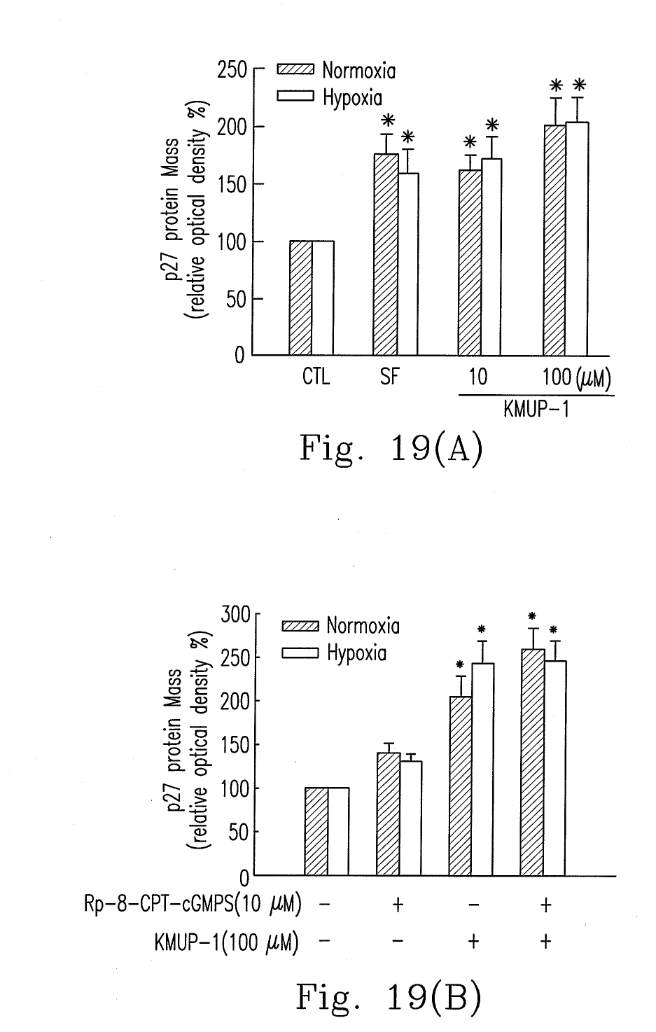

Please refer to FIGS. 19(A) and 19(B), which show the results of KMUP-1 (100 μM) on the expression of p27 in normoxia and hypoxia states after pretreatment with (FIG. 19B) or without (FIG. 19A) a cGMP antagonist, Rp-8-CPT-cGMP (10 μM) for 30 mins. As FIG. 19(A) shows, p27 expression is increased by KMUP-1 (10, 100 μM) as follows: 162.5±12.5%, 201.6±23.5% (normoxia) and 172.4±19.5%, 242.2±21.5% (hypoxia). However Rp-8-CPT-cGMPS (10 μM) also could not inhibit KMUP-1-induced p27 protein (FIG. 19B). Serum starvation of cells also shows the increased expression of p21 and p27.

In cell cycle, CDK-inhibitory proteins p21 and p27 are markers of DNA replication during cell progressing and are usually activated by p53 after DNA damage. Upon genotoxic damage, p21 and p27 contribute to cell-cycle arrest at the G0/G1 check points through diverse mechanism. KMUP-1 (10˜100 μM) inhibites the proliferation of cultured H441 cells in hypoxia, but shows no inhibition below 1.0 μM. However, growing evidence supports a role for p21 and p27 in regulation at translation level. KMUP-1 of the present invention increases the expression of p21 and p27 upon stress stimulation by hypoxia, indicating affecting the translation level during cancer cell growth. Moreover, KMUP-1-induced p21 and p27 expressions are unaffected by Rp-8-CPT-cGMPS, suggesting that cGMP-independent cell cycle progression is involved therein.

Bax/Bcl-2 and Caspase 3

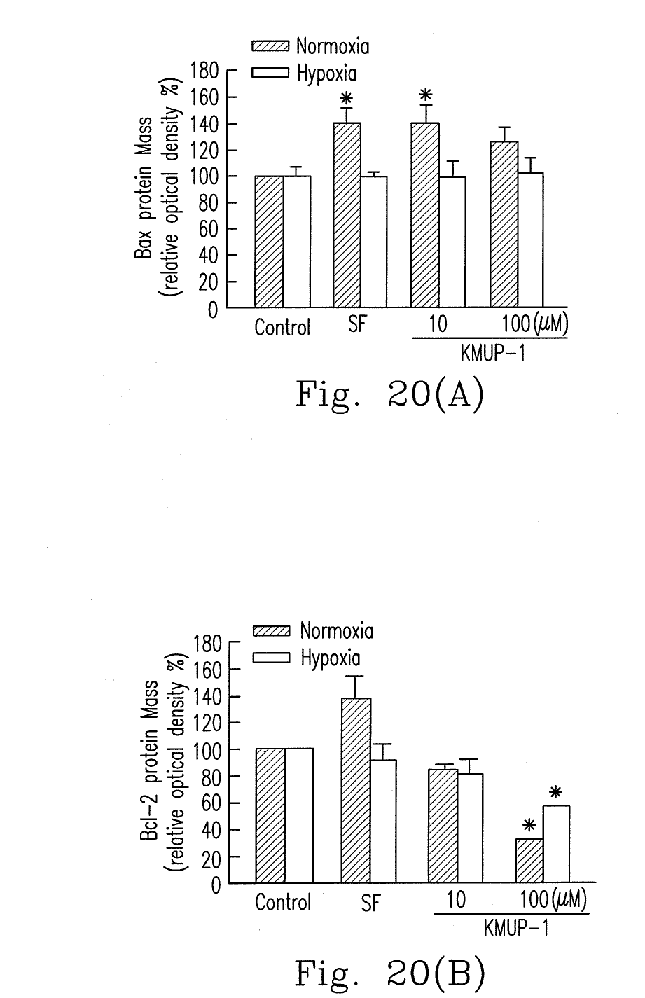

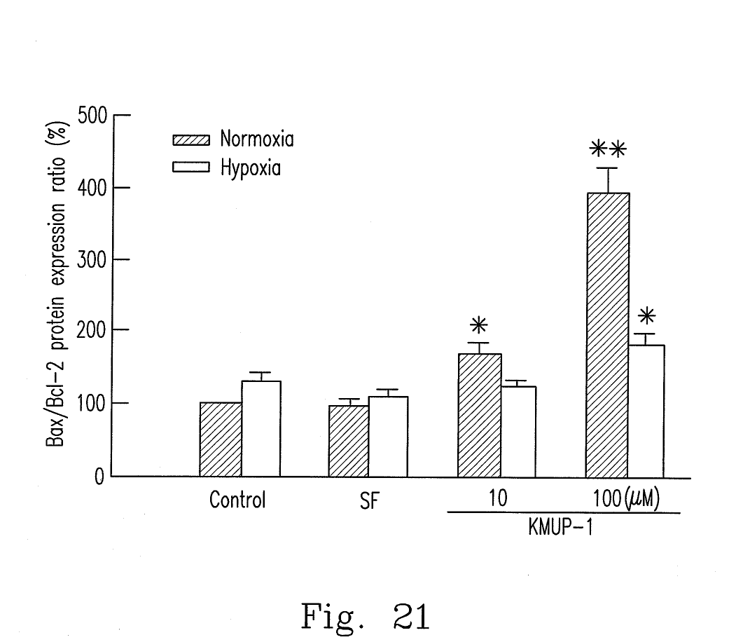

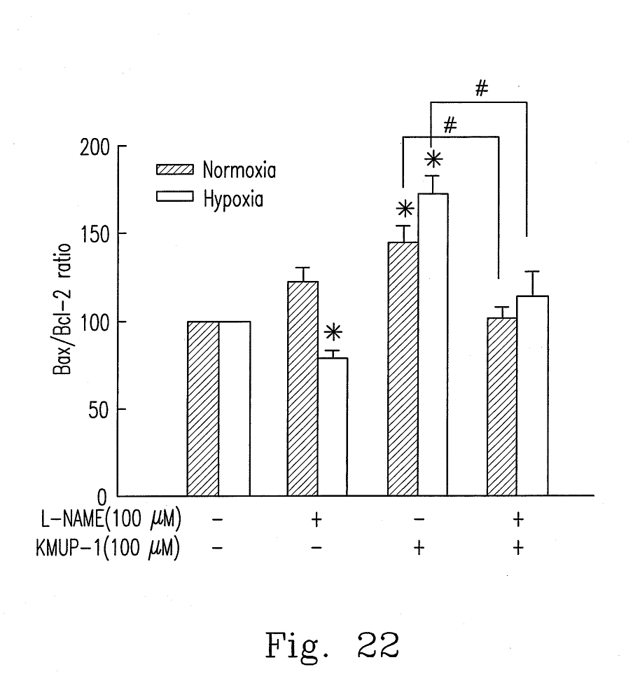

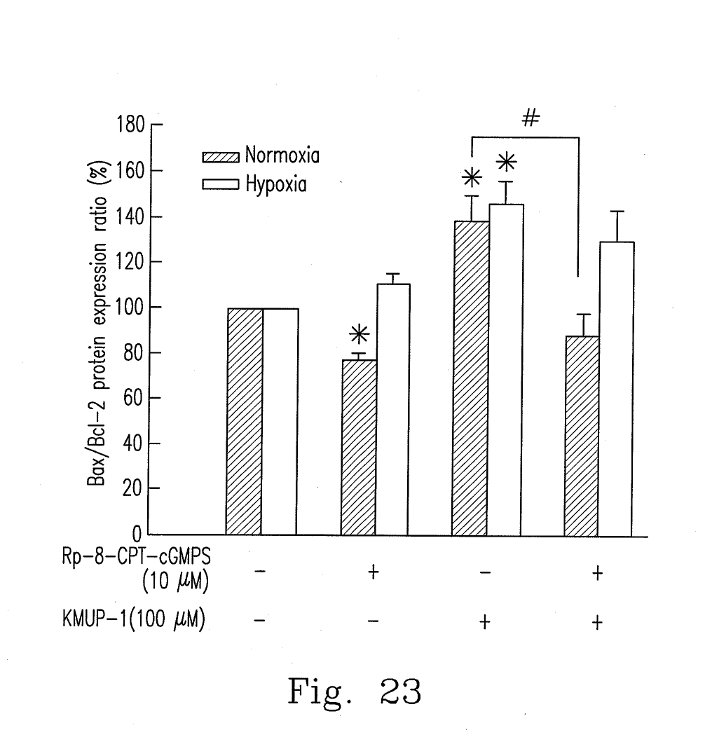

Please refer to FIGS. 20(A) and 20(B), which show the effects of KMUP-1 (10, 100 μM) on Bax (FIG. 20A) and Bcl-2 (FIG. 20B) expressions in H441 cells under normoxic and hypoxic conditions. KMUP-1 (10, 100 μM) increase Bax from control to 125.6±11.4% and 103.6±10.8%, respectively, and reduce Bcl-2 from control to 31.9±1.2% in normoxia and 56.5±1.3% in hypoxia. Accordingly, as shown in FIG. 21, Bax/Bcl-2 ratio increases with the concentrations of KMUP-1. As shown in FIGS. 22 and 23, L-NAME and Rp-8-CPT-cGMP inhibite the Bax/bcl-2 ratio increased by KMUP-1 (100 μM).

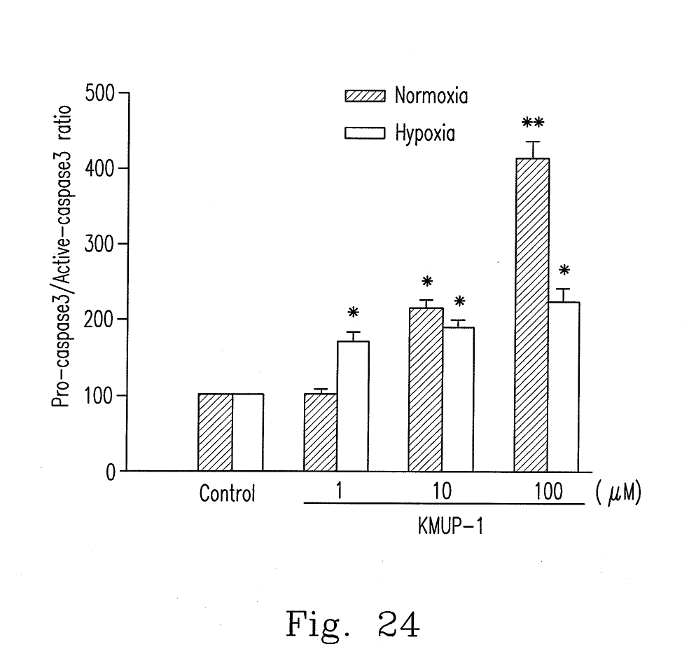

Please refer to FIG. 24, which shows the effects of KMUP-1 (1, 10, 100 μM) on procaspase-3/caspase-3 ratio in H441 cells under normoxic and hypoxic conditions, wherein the pro-caspase/active caspase 3 expression ratios are concentration-dependently increased, more in normoxia than in hypoxia.

According to the experiments of cell cycle in this invention, the appearance of a sub-G1 peak (apoptotic peak) is induced in H441 cells exposed to 72 hrs hypoxia, and the co-incubation with KMUP-1 (100 μM) enhances the apoptotic peak. KMUP-1 (10, 100 μM) also increases the Bax and decreases the Bcl-2 expressions in hypoxia, resulting in the increase of Bax/Bcl-2 ratio. Moreover, KMUP-1 enhances the caspase-3 expression, indicating apoptotic ability in H441 cells (FIG. 24)^{[11]}.

Noteworthily, KMUP-1 increases eNOS expression both in normoxia and in hypoxia, and thus theoretically would increase NO-mediated angiogenesis and toxic peroxynitrite (ONOO³¹ ). Additionally, KMUP-1-mediated Bax/Bcl-2 ratio is inhibited by a NOS inhibitor L-NAME and a PKG inhibitor Rp-8-CPT-cGMPS (FIGS. 22-23). Therefore, it is suggested that KMUP-1-caused H441 cells apoptosis could be due to NO-mediated overproduction of peroxynitrite via the Bax/Bcl-2 and cGMP-dependent pathways, in contrast to YC-1's cGMP-independent anti-proliferation in HA22T cell^{[12].}

U46619-Induced PDE-5A

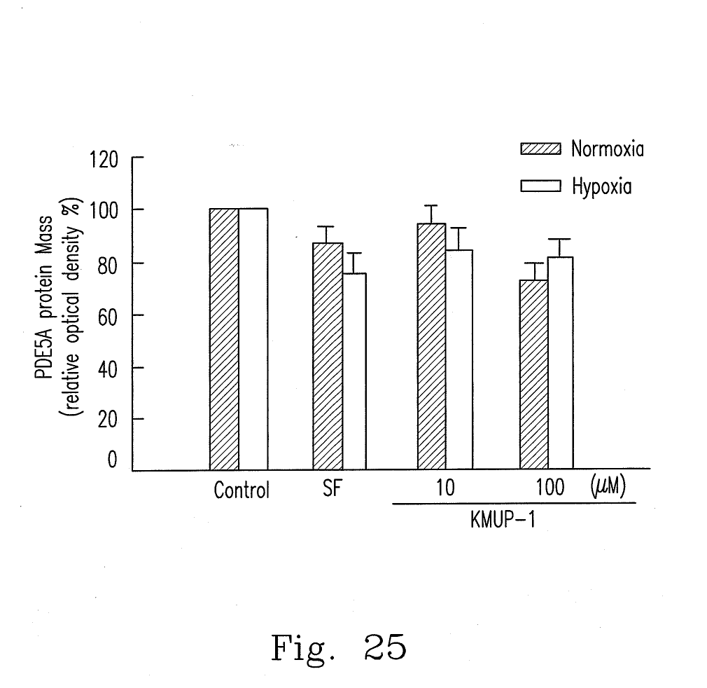

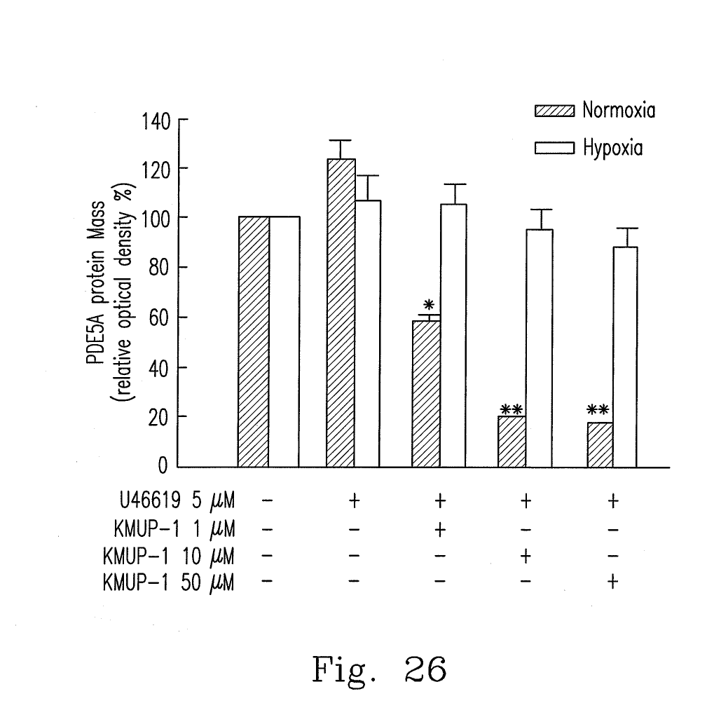

Please refer to FIG. 25, which shows the effects of KMUP-1 on PDE5A expression in H441 cells under normoxic and hypoxic conditions. Further refer to FIG. 26, which shows the effects of different concentrations of KMUP-1 (1.0, 10 and 100 μM) on the expression of PDE5A in H441 cells treated with a PDE5A enhansor, U46619 (5 μM), in normoxia and hypoxia states. As, FIG. 26 shows, inflammatory TXA2 mimetic agonist U46619 (5 μM) induces significant increase of PDE-5A expression in normoxia. However, pretreatments with KMUP-1 (1, 10, 50 μM), concentration-dependently decreases U46619-induced expression of epithelial PDE5A.

p38 Phpsphorylation and TNF-a-Induced iNOS

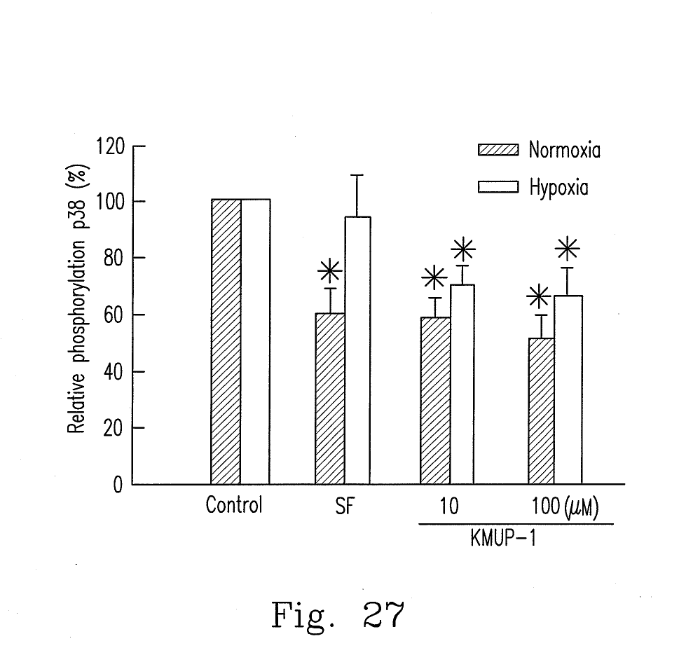

Please refer to FIG. 27, which shows the effects of KMUP-1 (10, 100 μM) on phosphate p-38/total p38 ratio in H441 cells under normoxic and hypoxic conditions. As FIG. 27 shows, the relative optical density ratio indicates the expression ratio of phosphate-p38/total p38. The expression ratio of the serum free group decreases in normoxia (n=4, p<0.05), but insignificantly increases in hypoxia. KMUP-1 (10, 100 μM) decreases the expression ratios to 58.9±7.1% and 50.9±9.1% in normoxia, 70.6±6.6% and 67.1±9.4% in hypoxia (n=4, p<0.05). It is believed that reduced phosphate-p38 expression by KMUP-1 indicates the potential in inhibiting pro-inflammatory pathway.

p38 kinase, a pro-inflammatory signaling protein, is believed to be a family member primarily responsible for regulation of inflammation. A variety of factors, including hypoxic stress, activates the expression of p38 kinase. Once activated, p38 phosphorylates downstream substrates to initiate a signal cascade that regulates synthesis of a variety of pro-inflammatory mediators. Regulation of p38 kinase by Rho/ROCK signaling has been described in vascular smooth muscle cell migration, which is sensitive to ROCK inhibitor Y27632. In the present invention, KMUP-1 attenuats ROCKII/p38 expression and inhibited cell migration both in normoxia and hypoxia, providing the anti-inflammation and anti-metastasis potential in lung epithelial cells.

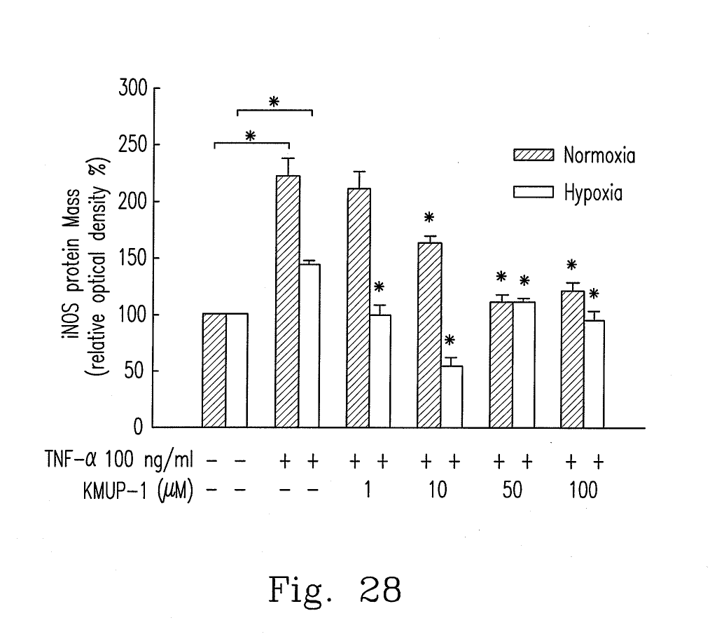

Please refer to FIG. 28, which shows the effects of different concentrations of KMUP-1 (1-100 μM) on the expression of TNF-a-induced iNOS in normoxia and hypoxia states. As, FIG. 28 shows, TNF-a (100 ng/ml) increases the expression of iNOS, and KMUP-1 (1, 10, 100 μM) further inhibites TNF-a-induced iNOS expressions in lung epithelial cell. TNFA-induced iNOS is attenuated by KMUP-1 and abolished at higher concentrations of KMUP-1 (≧50 μM) under normoxia and hypoxia, indicating anti-proinflammatory potent in both states.

In summary, KMUP-1 exhibits p38, ROCKII and VEGF inhibition, which has no cytotoxicly, promises the potential for pulmonary epithelium anti-proliferation, anti-proinflammation and anti-migration activities, and reveals applicability in preventing obstruction diseases caused by inflammation.

Accordingly, the present invention firstly shows that a cGMP-enhancing Rho-kinase inhibitor KMUP-1 might be used in the treatment of airway obstruction and proinflammation diseases and cancer cell metastasis, and thus it fits the demand of the industry and is industrially valuable.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

### REFERENCES

1. Zhan X., Li D., and Johns R. A. (1999) J. Histochem. Cytochem. 47, 1369-1374
2. Nijkamp F P, van der Linde H J, Folkerts G (1993) Am Rev Respir Dis. 148, 727-734
3. Wu, B. N., Chen, C. W., Liou, S. F, Yeh, J. L., Chung, H. H., and Chen, I. J. (2006) Molecular pharmacology 70 (3), 977-985.
4. Thompson, H. J., Jiang, C., Lu, J., Mehta, R. G., Piazza, G. A., Paranka, N. S., Pamukcu, R., and Ahnen, D. J. (1997) Cancer research 57(2), 267-271.
5. Soh, J. W., Mao, Y, Kim, M. G., Pamukcu, R., Li, H., Piazza, G. A., Thompson, W. J., and Weinstein, I. B. (2000) Clin Cancer Res 6(10), 4136-4141.
6. Yeo, E. J., Chun, Y. S., and Park, J. W. (2004) Biochemical pharmacology 68(6), 1061-1069.
7. Chun, Y. S., Yeo, E. J., Choi, E., Teng, C. M., Bae, J. M., Kim, M. S., and Park, J. W. (2001) Biochemical pharmacology 61(8), 947-954.
8. Nath, P., Leung, S. Y., Williams, A., Noble, A., Chakravarty, S. D., Luedtke, G. R., Medicherla, S., Higgins, L. S., Protter, A., and Chung, K. F. (2006) European journal of pharmacology 544(1-3), 160-167.
9. Yoshioka, Y, Yamamuro, A., and Maeda, S. (2006) Journal of pharmacological sciences 101(2), 126-134.
10. Deguchi, A., Thompson, W. J., and Weinstein, I. B. (2004) Cancer Res. 64, 3966-3973
11. Hakuma, N., Kinoshita, I., Shimizu, Y., Yamazaki, K., Yoshida, K., Nishimura, M., and Dosaka-Akita, H. (2005) Cancer Res. 65, 10776-10782
12. Zhu, B., Vemavarapu, L., Thompson, W. J., and Strada, S. J. (2005) J. Cell. Biochem. 94, 336-350

1-5. (canceled)

6. A method for inhibiting a physiological activity of a lung epithelial cell, comprising a step of:
administrating a pharmaceutically effective amount of a compound of 7-[2-[4-(2-chlorophenyl)piperazinyl]-ethyl]-1,3-dimethylxanthine (KMUP-1) to a mammal in need,
wherein the compound is a Rho-kinase inhibitor and being synthesized from xanthine, and the physiological activity is one selected from a group consisting of a proliferation activity, a migration activity, a pro-inflammatory activity and a combination thereof.

7. A method as claimed in claim 6, wherein the compound further comprises a pharmaceutically effective carrier.

8. (canceled)

9. A method as claimed in claim 6, wherein the migration activity is a metastasis activity of a cancer cell.

10. A method as claimed in claim 6, wherein the physiological activity is inhibited by cGMP-enhancing and ROCK inhibitory property of the compound.

11. A method for preparing a pharmaceutical composition, wherein the pharmaceutical composition has an inhibitory effect on one physiological activity of a lung epithelial cell selected from a group consisting of a proliferation, a migration, a pro-inflammatory and a combination thereof, and the pharmaceutical composition contains a compound of 7-[2-[4-(2-chlorophenyl)piperazinyl]-ethyl]-1,3-dimethylxanthine (KMUP-1).

12. (canceled)

13. A method as claimed in claim 11, wherein the migration activity is a metastasis activity of a cancer cell.

14. A method as claimed in claim 11, wherein the pharmaceutical composition further contains a pharmaceutically acceptable carrier.
